(19) United States
(12) Patent Application Publication
(10) Pub. No.: US 2025/0332580 A1
(43) Pub. Date: Oct. 30, 2025

(54) CATALYSTS AND RELATED METHODS OF MAKING AND USING THE SAME

(71) Applicant: NOVA CHEMICALS (INTERNATIONAL) S.A., Fribourg (CH)

(72) Inventors: Ce YANG, Clarksville, MD (US); Prashant KUMAR, Edmonton (CA); Elena SEBASTIAO, Calgary (CA); Hessam ZIAEIAZAD, Edmonton (CA)

(21) Appl. No.: 18/869,964

(22) PCT Filed: May 30, 2023
(86) PCT No.: PCT/IB2023/055526

§ 371 Date: Nov. 27, 2024

Related U.S. Application Data
(60) Provisional application No. 63/347,396, filed on May 31, 2022.

(51) Int. Cl. B01J 29/24; B01J 23/06; C07C 1/04; C07C 1/12

(52) U.S. Cl. CPC B01J 29/24; B01J 23/06; C07C 1/044; C07C 1/12

(57) ABSTRACT

Syngas conversion catalysts are H-MOR catalysts including iron and zinc. The catalysts can be made using a solid-state ion exchange process. The catalysts can be used in DSTO processes.

### TECHNICAL FIELD

The disclosure relates to catalysts and related methods of making and using such catalysts. In certain embodiments, the catalysts are syngas conversion catalysts.

### BACKGROUND ART

Catalysts, such as syngas conversion catalysts, have many different commercial uses. As an example, direct catalytic conversion of syngas to light olefins (DSTO), such as ethylene (C₂H₄), can be used as a step in certain commercial processes, such as advanced recycling of municipal solid waste (including plastic waste) via gasification technology.

### SUMMARY OF INVENTION

The disclosure provides catalysts and related methods of making and using such catalysts. In certain embodiments, the catalysts are syngas conversion catalysts.

In general, the catalysts are H-MOR catalysts that contain both iron (Fe) and zinc (Zn).

The catalysts can exhibit relatively high carbon monoxide (CO) conversion, relatively high light olefin (C₂-C₄) selectivity, and/or relatively low carbon dioxide (CO₂) selectivity.

The catalysts can be particularly beneficial when used in DSTO processes, including commercial DSTO processes. For example, when used in a DSTO process, the catalyst can form a relatively large amount of one or more desired products, such as ethylene, while forming a relatively small amount of one or more undesired products, such as carbon dioxide.

In some embodiments, the catalysts are made using a solid-state ion exchange process.

In a first aspect, the disclosure provides a catalyst that includes H-MOR, from 2.0 weight percent (wt %) to 6.5 wt % Fe, and from 0.1 weight percent to 2.0 wt % Zn.

In some embodiments, the catalyst includes from 0.2 wt % to 1.8 wt % Zn (e.g., from 0.3 wt % to 1.75 wt % Zn, from 0.4 wt % to 1.7 wt % Zn).

In some embodiments, the catalyst includes from 1.0 wt % to 6.0 wt % Fe (e.g., from 2.3 wt % to 5.8 wt % Fe, from 2.8 wt % to 5.6 wt % Fe).

In some embodiments, the catalyst has a CO₂ selectivity of at most 30% (e.g., a CO₂ selectivity of at most 25%, a CO₂ selectivity of at most 20%).

In some embodiments, the catalyst has a CO conversion of at least 40% (e.g., a CO conversion of at least 50%, a CO conversion of at least 60%).

In some embodiments, the catalyst has a selectivity for C₂-C₄ olefins of at least 20% (e.g., a selectivity for C₂-C₄ olefins of at least 25%, a selectivity for C₂-C₄ olefins of at least 30%).

In some embodiments, the catalyst has a Fe/Zn molar ratio from 2.0 and to 5.0 (e.g., a Fe/Zn molar ratio of from 3.0 to 4.5, a Fe/Zn molar ratio of from 3.7 to 4.4, a Fe/Zn molar ratio of from 3.9 to 4.2).

In a second aspect, the disclosure provides a catalyst that includes H-MOR, Fe and Zn, wherein the catalyst has a CO₂ selectivity of at most 30%.

In some embodiments, the catalyst has a CO₂ selectivity of at most 25% (e.g., a CO₂ selectivity of at most 20%, a CO₂ selectivity of at most 15%, a CO₂ selectivity of from 5% to 15%).

In some embodiments, the catalyst has a CO conversion of at least 40% (e.g., a CO conversion of at least 50%, a CO conversion of at least 60%).

In some embodiments, the catalyst has a selectivity for C₂-C₄ olefins of at least 20% (e.g., a selectivity for C₂-C₄ olefins of at least 25%, a selectivity for C₂-C₄ olefins of at least 35%).

In some embodiments, the catalyst has a Fe/Zn molar ratio of from 2.0 and to 5.0 (e.g., a Fe/Zn molar ratio of from 3.0 to 4.5, a Fe/Zn molar ratio of from 3.7 to 4.4, a Fe/Zn molar ratio of from 3.9 to 4.2).

In a third aspect, the disclosure provides a catalyst that includes H-MOR, Fe and Zn, wherein the catalyst has a CO conversion of at least 40%.

In some embodiments, the catalyst has a CO conversion of at least 50% (e.g., a CO conversion of at least 60%, a CO conversion of at least 70%, a CO conversion of at least 80%).

In some embodiments, the catalyst has a selectivity for C₂-C₄ olefins of at least 20% (e.g., a selectivity for C₂-C₄ olefins of at least 25%, a selectivity for C₂-C₄ olefins of at least 30%).

In some embodiments, the catalyst has a Fe/Zn molar ratio of from 2.0 and to 5.0 (e.g., a Fe/Zn molar ratio of from 3.0 to 4.5, a Fe/Zn molar ratio of from 3.7 to 4.4, a Fe/Zn molar ratio of from 3.9 to 4.2).

In a fourth aspect, the disclosure provides a catalyst that includes H-MOR, Fe and Zn, wherein the catalyst has a selectivity for C₂-C₄ olefins of at least 20% (e.g., a selectivity for C₂-C₄ olefins of at least 25%, a selectivity for C₂-C₄ olefins of at least 30%).

In some embodiments, the catalyst has a Fe/Zn molar ratio of from 2.0 and to 5.0 (e.g., a Fe/Zn molar ratio of from 3.0 to 4.5, a Fe/Zn molar ratio of from 3.7 to 4.4, a Fe/Zn molar ratio of from 3.9 to 4.2).

In a fifth aspect, the disclosure provides a catalyst that includes H-MOR, Fe and Zn, wherein the catalyst has a Fe/Zn molar ratio of from 2.0 to 5.0.

In some embodiments, the catalyst has a Fe/Zn molar ratio of from 3.0 to 4.5 (e.g., a Fe/Zn molar ratio of from 3.7 to 4.4, a Fe/Zn molar ratio of from 3.9 to 4.2).

In a sixth aspect, the disclosure provides a method that includes contacting a gas mixture including CO and CO₂ and a catalyst to form C₂-C₄ olefins, wherein the catalyst is a catalyst according to the disclosure.

In some embodiments, the gas mixture includes syngas.

In some embodiments, the gas mixture has a pressure of from 100 psig to 600 psig.

In some embodiments, the gas mixture has a temperature of from 200° C. to 450° C.

In some embodiments, a flow rate of the gas mixture is between 375 ml/h/g_{cat} and 6000 ml/h/g_{cat}.

In some embodiments, a flow rate of the gas mixture is between 100 h⁻¹ and 800 h⁻¹.

In some embodiments, a linear velocity of the gas is at least 1 cm/s. As used herein, the linear velocity of a gas is defined as the reactor inlet flow at conditions of standard temperature pressure divided by the product of the porosity fraction or voidage and the cross sectional area of the reactor tube.

In a seventh aspect, the disclosure provides a method that includes making a catalyst according to the disclosure.

In some embodiments, the method includes using solid-state ion exchange.

In some embodiments, the method includes combining X-MOR, an iron hydrate, and a zinc hydrate to provide a mixture, wherein X includes a cation. In some embodiments, at least one of the following holds: X includes NH₄⁺ ion; the iron hydrate includes FeCl₂·4H₂O; and the zinc hydrate includes Zn(NO₃)₂·6H₂O.

In some embodiments, the method further includes grinding the mixture to provide a powder.

In some embodiments, the method further includes heating the powder to a first temperature to provide an intermediate.

In some embodiments, at least one of the following holds: heating to the first temperature is performed in an inert gas atmosphere; the first temperature is at least 150° C. and/or high enough to melt the salts; and the first temperature is held for at least one hour.

In some embodiments, the method further includes heating the intermediate to a second temperature greater than the first temperature.

In some embodiments, at least one of the following holds: heating to the second temperature is performed in an inert gas inert atmosphere; the second temperature is at least 400° C.; and the second temperature is maintained for at least at least 4 hours.

In some embodiments, the temperature is increased from the first to the second at a rate of at least 1° C./minute.

In some embodiments, the inert atmosphere is a nitrogen atmosphere.

### BRIEF DESCRIPTION OF THE FIGURES

FIGS. 1A and 1B are tables showing experimental data.

FIGS. 2A-2C are tables showing experimental data.

### DESCRIPTION OF EMBODIMENTS

### Catalysts

Generally, a catalyst according to the disclosure is a H-MOR catalyst that includes iron and zinc.

As used herein, MOR is used as an abbreviation for Mordenite. As an example, H-Mordenite, where H⁺ is a counter ion, is referred to as H-MOR. As another example, Na-Mordenite, where Na⁺ is a counter ion, is referred to as Na-MOR. As a further example, NH₄-Mordenite, where NH₄⁺ is a counter ion, is referred to as NH₄-MOR. As an additional example, pyridine-Mordenite, where pyridine is impregnated, binding to H⁺, but not intact after calcination, is referred to as Py-MOR. As another example, Fe₄Zn—H-Mordenite is referred to as Fe₄Zn—H-MOR or Fe₄Zn-MOR.

In some embodiments, the amount Fe in the catalyst is 1.0-6.5 weight percent (wt %) (e.g., 1.0 wt %, 1.5 wt %, 2.0 wt %, 2.3 wt %, 2.5 wt %, 2.8 wt %, 3.0 wt %, 3.5 wt %, 4.0 wt %, 4.5 wt %, 5.0 wt %, 5.5 wt %, 5.6 wt %, 5.8 wt %, 6.0 wt %, 6.5 wt %, 1.0-6.0 wt %, 1.0-5.8 wt %, 1.0-5.6 wt %, 1.0-5.5 wt %, 1.0-5.0 wt %, 1.0-4.5 wt %, 1.0-4.0 wt %, 1.0-3.5 wt %, 1.0-3.0 wt %, 1.0-2.8 wt %, 1.0-2.5 wt %, 1.0-2.3 wt %, 1.0-2.0 wt %, 1.0-1.5 wt %, 1.5-6.0 wt %, 1.5-5.8 wt %, 1.5-5.6 wt %, 1.5-5.5 wt %, 1.5-5.0 wt %, 1.5-4.5 wt %, 1.5-4.0 wt %, 1.5-3.5 wt %, 1.5-3.0 wt %, 1.5-2.8 wt %, 1.5-2.5 wt %, 1.5-2.3 wt %, 1.5-2.0 wt %, 2.0-6.0 wt %, 2.0-5.8 wt %, 2.0-5.6 wt %, 2.0-5.5 wt %, 2.0-5.0 wt %, 2.0-4.5 wt %, 2.0-4.0 wt %, 2.0-3.5 wt %, 2.0-3.0 wt %, 2.0-2.8 wt %, 2.0-2.5 wt %, 2.0-2.3 wt %, 2.3-6.5 wt %, 2.3-6.0 wt %, 2.3-5.8 wt %, 2.3-5.6 wt %, 2.3-5.5 wt %, 2.3-5.0 wt %, 2.3-4.5 wt %, 2.3-4.0 wt %, 2.3-3.5 wt %, 2.3-3.0 wt %, 2.3-2.8 wt %, 2.3-2.5 wt %, 2.5-6.5 wt %, 2.5-6.0 wt %, 2.5-5.8 wt %, 2.5-5.6 wt %, 2.5-5.5 wt %, 2.5-5.0 wt %, 2.5-4.5 wt %, 2.5-4.0 wt %, 2.5-3.5 wt %, 2.5-3.0 wt %, 2.5-2.8 wt %, 2.8-6.5 wt %, 2.8-6.0 wt %, 2.8-5.8 wt %, 2.8-5.6 wt %, 2.8-5.5 wt %, 2.8-5.0 wt %, 2.8-4.5 wt %, 2.8-4.0 wt %, 2.8-3.5 wt %, 2.8-3.0 wt %, 3.0-6.5 wt %, 3.0-6.0 wt %, 3.0-5.8 wt %, 3.0-5.6 wt %, 3.0-5.5 wt %, 3.0-5.0 wt %, 3.0-4.5 wt %, 3.0-4.0 wt %, 3.0-3.5 wt %, 3.5-6.5 wt %, 3.5-6.0 wt %, 3.5-5.8 wt %, 3.5-5.6 wt %, 3.5-5.5 wt %, 3.5-5.0 wt %, 3.5-4.5 wt %, 3.5-4.0 wt %, 4.0-6.5 wt %, 4.0-6.0 wt %, 4.0-5.8 wt %, 4.0-5.6 wt %, 4.0-5.5 wt %, 4.0-5.0 wt %, 4.0-4.5 wt %, 4.5-6.5 wt %, 4.5-6.0 wt %, 4.5-5.8 wt %, 4.5-5.6 wt %, 4.5-5.5 wt %, 4.5-5.0 wt %, 5.0-6.5 wt %, 5.0-6.0 wt %, 5.0-5.8 wt %, 5.0-5.6 wt %, 5.0-5.5 wt %, 5.5-6.5 wt %, 5.5-6.0 wt %, 5.5-5.8 wt %, 5.8-6.5 wt %, 5.8-6.0 wt %, 6.0-6.5 wt %).

In certain embodiments, the wt % of Zn in the catalyst is 0.1-2.0 wt % (e.g., 0.1 wt %, 0.2 wt %, 0.3 wt %, 0.4 wt %, 0.5 wt %, 0.6 wt %, 0.7 wt %, 0.8 wt %, 0.9 wt %, 1.0 wt %, 1.1 wt %, 1.2 wt %, 1.3 wt %, 1.4 wt %, 1.5 wt %, 1.6 wt %, 1.7 wt %, 1.75 wt %, 1.8 wt %, 1.9 wt %, 2.0 wt %, 0.1-1.9 wt %, 0.1-1.8 wt %, 0.1-1.75 wt %, 0.1-1.7 wt %, 0.1-1.6 wt %, 0.1-1.5 wt %, 0.1-1.4 wt %, 0.2-1.9 wt %, 0.2-1.8 wt %, 0.2-1.75 wt %, 0.2-1.7 wt %, 0.2-1.6 wt %, 0.2-1.5 wt %, 0.2-1.4 wt %, 0.3-1.9 wt %, 0.3-1.8 wt %, 0.3-1.75 wt %, 0.3-1.7 wt %, 0.3-1.6 wt %, 0.3-1.5 wt %, 0.3-1.4 wt %, 0.4-1.9 wt %, 0.4-1.8 wt %, 0.4-1.75 wt %, 0.4-1.7 wt %, 0.4-1.6 wt %, 0.4-1.5 wt %, 0.4-1.4 wt %, 0.45-1.9 wt %, 0.45-1.8 wt %, 0.45-1.75 wt %, 0.45-1.7 wt %, 0.45-1.6 wt %, 0.45-1.5 wt %, 0.45-1.4 wt %).

In some embodiments, the catalyst according to the disclosure can have an Fe/Zn molar ratio of from 2.0-5.0 (e.g., 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 2.0-5.0, 2.0-4.5, 2.0-4.4, 2.0-4.2, 2.0-3.75, 2.0-3.5, 2.0-3.25, 2.0-3.0, 2.0-2.5, 2.5-5.0, 2.5-4.5, 2.5-4.4, 2.5-4.2, 2.5-3.75, 2.5-3.5, 2.5-3.25, 2.5-3.0, 3.0-5.0, 3.0-4.5, 3.0-4.4, 3.0-4.2, 3.0-3.75, 3.0-3.5, 3.0-3.25, 3.25-5.0, 3.25-4.5, 3.25-4.4, 3.25-4.2, 3.25-3.75,3.25-5.0, 3.25-4.5, 3.25-4.4, 3.25-4.2, 3.25-3.75, 3.25-3.5, 3.75-5.0, 3.75-4.5, 3.75-4.4, 3.75-4.2, 3.8-5.0, 3.8-4.5, 3.8-4.4, 3.8-4.2, 3.9-5.0, 3.9-4.5, 3.9-4.4, 3.9-4.2).

In some embodiments, the catalyst according to the disclosure can have a relatively high CO conversion. As used herein, the CO conversion is calculated as

$${x}_{CO} ( \% ) = \frac{{n}_{CO} , in - {n}_{CO} , out}{{n}_{CO} , in} \times 100$$

as measured at 15 hours of the catalyst on the syngas stream composed of 60% by volume H₂, 30% by volume CO and 10% by volume CO₂, at a temperature of 350-380° C., a pressure of 300-400 psig, and a gas hourly space velocity (GHSV) of 1050-1500 ml/h/g_{cat} for syngas with a H₂/CO ratio of 2 or more. n_{CO}, in is the moles of CO input. n_{CO}, out is the moles of CO output.

In some embodiments, the catalyst according to the disclosure has a CO conversion of at least 40% (e.g., at least 50%, at least 60%, at least 70%, at least 80%) and at most 90% (e.g., at most 80%, at most 70%, at most 60%). In certain embodiments, the catalyst according to the disclosure has a CO conversion of 40%-90% (e.g., 50%-90%, 60%-90%, 70%-90%, 80%-90%, 90%-90%, 40%-90%, 50%-90%, 60%-90%, 70%-90%, 80%-90%, 40%-80%, 50%-80%, 60%-80%, 70%-80%, 40%-70%, 50%-70%, 60%-70%, 40%-60%, 50%-60%).

In certain embodiments, the catalyst according to the disclosure can have a relatively low CO₂ selectivity. As used herein, the selectivity for CO₂ is calculated as

$${S}_{{CO}_{2}} ( \% ) = \frac{{n}_{CO 2} , out - {n}_{CO 2} , in}{{n}_{CO} , in - {n}_{CO} , out} \times 100$$

as measured at 15 hours of the catalyst on the syngas stream composed of H₂, CO and CO₂, at a temperature of 350-380° C., a pressure of 300-400 psig, and a gas hourly space velocity (GHSV) of 1050-1500 ml/h/g_{cat} for syngas with a H₂/CO ratio of 2 or more. n_{CO2}, out is the moles of CO₂ output. n_{CO2}, in is the moles of CO₂ input. n_{CO}, in is the moles of CO input. n_{CO}, out is the moles of CO output. In some embodiments, the catalyst according to the disclosure has selectivity for CO₂ of at most 30% (e.g., at most 25%, at most 20%, at most 15%, at most 10%) and at least 1% (e.g., at least 3%, at least 5%). In some embodiments, the catalyst according to the disclosure has a CO₂ selectivity of from 5-30% (e.g., 1%-30%, 1%-25%, 1%-20%, 1%-15%, 1%-10%, 3%-30%, 3%-25%, 3%-20%, 3%-15%, 3%-10%, 5%-30%, 5%-25%, 5%-20%, 5%-15%, 5%-10%).

In some embodiments, the catalyst according to the disclosure can have a relatively high selectivity for C₂-C₄ olefins. As used herein, a selectivity for C₂-C₄ olefins is calculated as

$${S}_{{c}_{2} - {c}_{4} =} ( \% ) = \frac{{n}_{{C}_{2} {H}_{4}} + {n}_{{C}_{3} {H}_{6}} + {n}_{{C}_{4} {H}_{8}}}{{n}_{CO} , in - {n}_{CO} , out} \times 100$$

as measured at 15 hours of the catalyst on the syngas stream composed of H₂, CO and CO₂, at a temperature of 350-380° C., a pressure of 300-400 psig, and a gas hourly space velocity (GHSV) of 1050-1500 ml/h/g_{cat} for syngas of H₂/CO ratio of 2 or more. n_{CO}, in is the moles of CO input. n_{CO}, out is the moles of CO output. n_{C}_{₂}_{H}_{₄} is the moles of C₂H₄ output. n_{C}_{₃}_{H}_{₆} is the moles of C₃H₆ output. n_{C}_{₄}_{H}_{₈} is the moles of C₄H₈ output.

In some embodiments, the catalyst according to the disclosure has a selectivity for C₂-C₄ olefins of at least 20% (e.g. at least 25%, at least 30%, at least 35%, at least 40%) and at most 95% (e.g., at most 75%, at most 50%). In certain embodiments, the catalyst according to the disclosure has a selectivity for C₂-C₄ olefins of 20%-95% (e.g., 20%-75%, 20%-50%, 25%-95%, 25%-75%, 25%-50%, 30-95%, 30%-75%, 30%-50%, 35%-95%, 35%-75%, 35%-50%, 40-95%, 40%-75%, 40%-50%).

In certain embodiments, the GHSV is calculated in h⁻¹.

In certain embodiments, the GHSV is calculated in ml/h/g_{cat}.

### Methods of Making Catalysts

In some embodiments, the catalyst according to the disclosure is made using a solid-state ion exchange process. Examples of making the catalyst according to the disclosure include adding Fe and Zn to NH₄-MOR and forming the catalyst via a solid-ion exchange process. In some embodiments, Na-MOR can be converted to NH₄-MOR using ion exchange reactions. In general, the Fe and Zn can be in any appropriate form. In some embodiments, Zn is in the form of a salt (e.g., Zn(NO₃)₂·6H₂O, Zn(OAc)₂·6H₂O), and/or Fe is in the form of a Fe salt (e.g., FeCl₂·4H₂O, Fe(NO₃)₂·9H₂O, FeSO₄·7H₂O). Generally, the Fe, Zn and NH₄-MOR are ground together using any appropriate method to provide a ground powder. In certain embodiments, grinding is achieved using a mortar and pestle. In some embodiments, the ground powder is heated to a first temperature to melt the salts. In general, any appropriate temperature can be used as the first temperature. In some embodiments, the first temperature is from 130° C. to 170° C. (e.g., 130° C., 140° C., 150° C., 160° C., 170° C., 140° C. to 160° C., 140° C. to 170° C.). Generally, after heating to the first temperature, the resulting composition is heated to a second temperature, which is higher than the first temperature. In certain embodiments, the second temperature is a temperature is from 400° C. to 600° C. (e.g., 400° C., 500° C., 600° C., 400° C. to 500° C., 500° C. to 600° C.). In general, the composition can be held at the second temperature for any appropriate period of time. In some embodiments, the composition is held at the second temperature for at least 3 hours (e.g. at least 4 hours, at least 5 hours, at least 6 hours). Generally, the composition can be heated from the first temperature to the second temperature using any appropriate temperature ramp rate. In some embodiments, the temperature ramp rate for heating the composition from the first temperature to the second temperature is at least 1.0° C./min (e.g., 1.0° C./min, 1.3° C./min, 1.5° C./min, 1.6° C./min, 1.7° C./min). In some embodiments, the heating is performed under an inert atmosphere. In some embodiments, the inert atmosphere is a nitrogen atmosphere.

### Methods of Using Catalysts

In some embodiments, the catalyst according to the disclosure is used in a DSTO process.

In certain embodiments, the catalyst according to the disclosure is used to convert a gas mixture containing CO, hydrogen (H₂) and optionally one or more gases (e.g., CO₂) to one or more hydrocarbons. An example of such a gas is syngas. Examples of such hydrocarbons include CH₄, C₂H₄, C₃H₆, C₄H₈, C₂H₆, C₃H₈, C₄H₁₀, C₂H₂, C₅₊. Typically, the conversion also results in the formation of one or more oxygen containing carbon compounds, such as methanol and dimethyl ether. Generally, any appropriate reaction conditions can be used to promote the conversion. In some embodiments the gas mixture has a pressure from 100 psig to 600 psi (e.g. 100 psig, 150 psig, 200 psig, 250 psig, 300 psig, 350 psig, 400 psig, 450 psig, 500 psig, 550 psig, 600 psig, 50-600 psig, 100-600 psig, 150-600 psig, 200-600 psig, 250-600 psig, 300-600 psig, 350-600 psig, 400-600 psig, 450-600 psig, 500-600 psig, 550-600 psig, 5-550 psig, 50-550 psig, 100-550 psig, 150-550 psig, 200-550 psig, 250-550 psig, 300-550 psig, 350-550 psig, 400-550 psig, 450-550 psig, 500-550 psig, 5-500 psig, 50-500 psig, 100-500 psig, 150-500 psig, 200-500 psig, 250-500 psig, 300-500 psig, 350-500 psig, 400-500 psig, 450-500 psig, 5-450 psig, 50-450 psig, 100-450 psig, 150-450 psig, 200-450 psig, 250-450 psig, 300-450 psig, 350-450 psig, 400-450 psig, 5-400 psig, 50-400 psig, 100-400 psig, 150-400 psig, 200-400 psig, 250-400 psig, 300-400 psig, 350-400 psig, 5-350 psig, 50-350 psig, 100-350 psig, 150-350 psig, 200-350 psig, 250-350 psig, 300-350 psig, 5-300 psig, 50-300 psig, 100-300 psig, 150-300 psig, 200-300 psig, 250-300 psig, 5-250 psig, 50-250 psig, 100-250 psig, 150-250 psig, 200-250 psig, 5-200 psig, 50-200 psig, 100-200 psig, 150-200 psig, 5-150 psig, 50-150 psig, 100-150 psig, 5-100 psig, 50-100 psig, 5-50 psig). In some embodiments, the gas mixture has a temperature of 200° C. to 400° C. (e.g. 200° C., 250° C., 300° C., 350° C., 400° C., 200-350° C., 200-300° C., 200-250° C., 250-400° C., 250-350° C., 250-300° C., 300-400° C., 300-350° C., 350-400° C.). In some embodiments, the GHSV of the gas is from 375 ml/h/g_{cat} and 6000 ml/h/g_{cat} (e.g. 375-5000 ml/h/g_{cat}, 375-4500 ml/h/g_{cat}, 375-4000 ml/h/g_{cat}, 375-3500 ml/h/g_{cat}, 375-3000 ml/h/g_{cat}, 375-2500 ml/h/g_{cat}, 375-2000 ml/h/g_{cat}, 375-1500 ml/h/g_{cat}, 375-1000 ml/h/g_{cat}, 1000-5000 ml/h/g_{cat}, 1000-4500 ml/h/g_{cat}, 1000-4000 ml/h/g_{cat}, 1000-3500 ml/h/g_{cat}, 1000-3000 ml/h/g_{cat}, 1000-2500 ml/h/g_{cat}, 1000-2000 ml/h/g_{cat}, 1000-1500 ml/h/g_{cat}, 1500-5000 ml/h/g_{cat}, 1500-4500 ml/h/g_{cat}, 1500-4000 ml/h/g_{cat}, 1500-3500 ml/h/g_{cat}, 1500-2500 ml/h/g_{cat}, 1500-2000 ml/h/g_{cat}, 2000-5000 ml/h/g_{cat}, 2000-4500 ml/h/g_{cat}, 2000-4000 ml/h/g_{cat}, 2000-3500 ml/h/g_{cat}, 2000-3000 ml/h/g_{cat}, 2000-2500 ml/h/g_{cat}, 2500-5000 ml/h/g_{cat}, 2500-4500 ml/h/g_{cat}, 2500-4000 ml/h/g_{cat}, 2500-3500 ml/h/g_{cat}, 2500-3000 ml/h/g_{cat}, 3000-5000 ml/h/g_{cat}, 3000-4500 ml/h/g_{cat}, 3000-4000 ml/h/g_{cat}, 3000-3500 ml/h/g_{cat}, 3500-5000 ml/h/g_{cat}, 3500-4500 ml/h/g_{cat}, 3500-4000 ml/h/g_{cat}, 4000-5000 ml/h/g_{cat}, 4000-4500 ml/h/g_{cat}, 4500-5000 ml/h/g_{cat}). In some embodiments, the gas has a linear velocity of at least 1 cm/s (e.g. at least 1.5 cm/s, at least 2 cm/s, at least 2.5 cm/s, at least 3 cm/s, at least 3.5 cm/s, at least 4 cm/s, at least 4.5 cm/s at least 5 cm/s). In some embodiments, the temperature is from 350° C. to 380° C., the pressure is from 300 psig to 400 psig, the GHSV of the syngas is between 1050-1500 mL/h/g_{cat}, and the CO₂ is co-fed of 10%. In certain embodiments, the catalyst is activated in the presence of H₂ prior to use. In some embodiments, the activation conditions are at least one of 10% H₂ in Ar, a temperature of 380-450° C., a time of 2-15 hours (e.g. 2-10 hours, 2-5 hours, 2-3 hours), and a pressure of atmospheric pressure.

### EXAMPLES

### Synthetic Equipment

### Split Tube Calcination Furnace

The split tube calcination furnace (Thermcraft TSP-1.63-0-8-2C-J7981/1A) was used to calcine samples of 10 g or less in a nitrogen gas atmosphere. The furnace temperature was controlled by a controller (Thermcraft 2-1-10-115-Y02SK-J7981) which has a user-defined ramping rate and a maximum temperature of 1010° C. The unit was equipped with secondary over-temperature protection.

A horizontal quartz tube (55 cm in length, 2.435 cm ID) sat within the furnace. The quartz tube was equipped with ground glass ball joints at each end which were connected to metal adaptors via clamps and silicon grease. The inlet of the quartz tube was connected to an apparatus where the selected gas could optionally be directed through various purification beds to remove oxygen and moisture prior to flowing through the tube. The outlet of the quartz tube was connected to a mineral oil trap followed by a vent. During operation, the quartz tube was purged constantly with low pressure gas at a flow ranging 10-100 sccm. When loaded with a sample, the quartz tube was generally purged at a flow rate of 85 sccm prior to starting the heat profile, while the calcination was generally executed with a flow rate of 30 sccm. The operational pressure was less than 4 psig.

### Quartz Reactor Unit 2 (QRU2)

The QRU2 (Lindberg Furnace, Model #54579-S) was used to calcine samples of 100 g or less in a nitrogen gas atmosphere or in air. The furnace temperature was controlled by three controllers (Lindberg Furnace Controller, Model #58475-P-B-2ALS and Eurotherm 847, 2404 Temperature Controllers) with user-defined ramping rates and a maximum temperature of 1500° C. The unit was equipped with a Honeywell Experion PKS DCS System and a Brooks Mass Flow Controller, Model #5850E.

A horizontal quartz tube (152.4 cm in length, 5.08 cm ID) sat within the furnace. The quartz tube was equipped with ground glass ball joints at each end which were connected to metal adaptors via clamps and silicon grease. The inlet of the quartz tube was connected to an apparatus where the nitrogen gas could optionally be directed through various purification beds to remove oxygen and moisture prior to flowing through the tube. The outlet of the quartz tube was connected to a series of three 1M HCl traps followed by a vent. During operation, the quartz tube was purged constantly with low-pressure gas at a flow ranging 10-1000 sccm. Once loaded with a sample, the quartz tube was generally purged at a flow rate of 400 sccm prior to starting the heat profile, while the calcination was generally executed with a flow rate of 400 sccm. The operational pressure was less than 4 psig.

### Muffle Furnace

The muffle furnace (Moldatherm Box Model 51894) was used to calcine samples of 100 g or less in air. The furnace had a built-in controller with user-defined ramping rate and a maximum temperature of 1100° C. The unit was equipped with secondary over-temperature protection.

### Materials

Sodium mordenite (Na-MOR), product number CBV 10, was purchased from ZEOLYST International. Ammonium chloride (NH₄Cl), product number 09718 BioUltra, ≥99.5%; zinc nitrate hexahydrate (Zn(NO₃)₂·6H₂O), product number 228737 reagent grade, 98%; iron(II) chloride tetrahydrate (FeCl₂·4H₂O), product number 220299 ReagentPlus®, 98%; chromium (III) nitrate nonahydrate (Cr(NO₃)₃·9H₂O), product number 239259, 99%; aluminum nitrate nonahydrate (Al(NO₃)₃·9H₂O), product number 237973 ACS reagent, ≥98%; pyridine, product number 270970 anhydrous, 99.8%; zinc acetate dihydrate (Zn(OAc)₂·2H₂O), product number 379786, 99.999%; iron(III) nitrate nonahydrate (Fe(NO₃)₃·9H₂O), product number 254223, ≥99.95%; iron(II) sulfate heptahydrate (Fe(SO₄·7H₂O), product number 215422 ≥99.95%; were all purchased from Sigma Aldrich. All chemicals were used without further purification, with the exception of pyridine. Pyridine was degassed using the freeze-pump-thaw procedure and cannula transferred into a Kontes flasks containing dried molecular sieves using Schlenk line techniques. The pyridine was stored dry under nitrogen atmosphere and over a bed of molecular sieves. Distilled water was obtained in-house from a Corning MP-12A Water Still (Mega Pure 12A Water Still: Model 12 Litre Auto MP-12A, Serial #230). Deionized water was supplied to the distillation apparatus via Petwa deionizing cylinders.

### Example 1: Synthesis of Py-MOR+CrZnAl Oxide

Na-MOR was converted to NH₄-MOR through three consecutive aqueous ions exchange reactions. Two 10-g batches were executed in parallel. To two 1-L round bottom flasks (RBFs) was charged 10 g of Na-MOR and 500 mL of 1 M ammonium chloride (NH₄Cl) solution. The contents of the RBFs were stirred at 80° C. for 3 hours. Each solution was then filtered into their own respective Buchner funnel containing three qualitative filter papers. Each filter cake was washed with 500 mL of distilled water and dried in an oven at 90° C. overnight. Yields were 7.5 and 8.0 g respectively. To two 1-L RBFs was charged 7.5 g and 8.0 g of Na/NH₄-MOR with 375 mL and 400 mL of 1 M NH₄Cl solution respectively. The contents of the RBFs were stirred at 80° C. for 3 hours. Both solutions were then filtered into a single Buchner funnel containing three qualitative filter papers. The filter cake was washed with 500 mL of distilled water and dried in an oven at 90° C. overnight. There was 12.86 g of material recovered. To a 1-L RBF was loaded 12.86 g of Na/NH₄-MOR with mL of 1 M NH₄Cl solution. The contents of the RBF were stirred at 80° C. for 3 hours. The solution was then filtered into a Buchner funnel containing three qualitative filter papers. The filter cake was washed with 500 mL of distilled water and dried in an oven at 90° C. overnight. There was 12.57 g of material recovered.

The NH₄-MOR was converted to H-MOR through calcination in air. The NH₄-MOR was loaded into a ceramic bowl and calcined using the muffle furnace. The furnace was ramped to 500° C. within 1 hour and held at 500° C. for 6 hours.

The H-MOR was impregnated with pyridine using a vacuum distillation setup. H-MOR was placed in a Kontes flask and evacuated to −30 mmHg at 210° C. for 4 hours in a vacuum oven. The sample continued to be evacuated overnight and the oven was cooled back down to room temperature. The H-MOR Kontes was then connected to a vacuum distillation arm on a Schlenk line, with a second Kontes containing pyridine over molecular sieves connected at the other end of the arm. The H-MOR was further vacuum dried (<100 mTorr) as the pyridine Kontes was degassed using the freeze-pump-thaw procedure (repeated 3 times). The vacuum distillation arm was isolated under static vacuum, and the pyridine was allowed to thaw. The pyridine vapor was then transferred to the H-MOR Kontes, such that the entire sample was submerged in liquid. The H-MOR was submerged in pyridine for 30 minutes. Use of water heating bath under the pyridine Kontes and a cold bath under the H-MOR Kontes facilitated the transfer. Excess pyridine was transferred back to the pyridine Kontes by use of a heating bath under the H-MOR Kontes. The final consistency of the H-MOR powder was free-flowing in small granular clumps. The H-MOR Kontes was sealed under vacuum and transferred to a glove box for the sample to equilibrate overnight, remaining sealed and under reduced atmosphere. The sample was removed from the glovebox and transferred into a quartz boat. The boat was loaded into the split tube and purged with purified nitrogen for one hour with a flow rate of 85 sccm. The flow rate was reduced to 30 sccm and the sample was calcined under purified nitrogen at 500° C. for 4 hours with a 1-h ramp. There was 3.55 g of catalyst recovered after calcination and the material was an off-white color. The Py-MOR powder was pressed with 12 metric tons using a 1-inch die set, crushed using a mortar and pestle, then sieved to 500-710 μm.

CrZnAl oxide was synthesized according to a co-precipitation reaction as described in Jiao, F., et al. (2016). Science 351(6277): 1065 and Jiao, F., et al. (2018). Angewandte Chemie International Edition 57(17): 4692-4696. Zn(NO₃)₂·6H₂O (29.12 g, 97.89 mmol), Cr(NO₃)₂·9H₂O (11.2 g, 27.99 mmol), and Al(NO₃)₃·9H₂O (10.5, 27.99 mmol) were dissolved in 200 mL of distilled water in a 500-mL RBF. The solution was heated and stirred in 70° C. oil bath. To the mixture was added dropwise 84 mL of 1 M (NH₄)₂CO₃ solution. The reaction was allowed to stir for 3 hours at 70° C. The solution was then filtered into a Buchner funnel containing three qualitative filter papers. The filter cake was washed with 2 L of distilled water and dried in an oven at 90° C. overnight. There was 8.56 g of material recovered, which had a light grey-blue color. The material was loaded into a ceramic bowl and calcined in the muffle furnace. The furnace was ramped to 500° C. within 30 minutes and held at 500° C. for 1 hour. There was 4.58 g of dark grey powder recovered after calcination. The CrZnAl oxide powder was pressed with 12 metric tons using a 1-inch die set, crushed using a mortar and pestle, then sieved to 500-710 μm.

The Py-MOR pressed material and the CrZnAl oxide pressed material were combined in a 1:1 ratio by weight in a bottle. The bottle was shaken to obtain a homogeneous distribution of the particles prior to loading on the fixed-bed syngas converter unit.

### Example 2: Synthesis of FeZn₍ₛ₎—H-MOR—1

Na-MOR was converted to NH₄-MOR through three consecutive aqueous ions exchange reactions. To a 2-L RBF was loaded 30 g of Na-MOR and 1.5 L of 1 M NH₄Cl solution. The contents of the RBF were stirred at 80° C. for 3 hours. The solution was then filtered into a Buchner funnel containing three qualitative filter papers. The filter cake was washed with 500 mL of distilled water and dried in an oven at 90° C. overnight. There was 26.46 g of material recovered. To a 2-L RBF was loaded 26.46 g of Na/NH₄-MOR and 1.325 L of 1 M NH₄Cl solution. The contents of the RBF were stirred at 80° C. for 3 hours. The solution was then filtered into a Buchner funnel containing three qualitative filter papers. The filter cake was washed with two 500-mL portions of distilled water and dried in an oven at 90° C. overnight. There was 25.48 g of material recovered. To a 2-L RBF was loaded 25.48 g of Na/NH₄-MOR and 1.275 L of 1 M NH₄Cl solution. The contents of the RBF were stirred at 80° C. for 3 hours. The solution was then filtered into a Buchner funnel containing three qualitative filter papers. The filter cake was washed with three 500-mL portions of distilled water and dried in an oven at 90° C. overnight. The material was left uncalcined as NH₄-MOR.

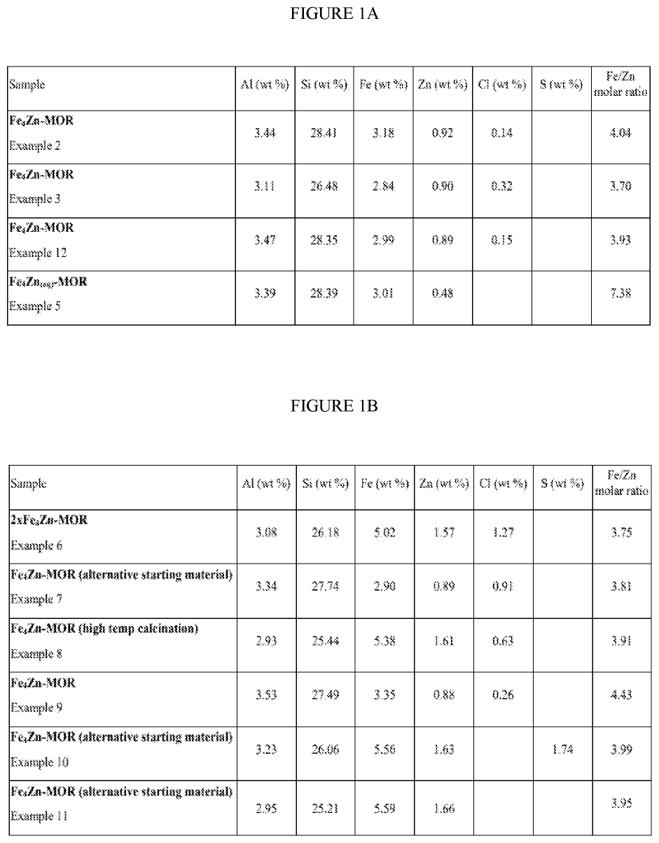

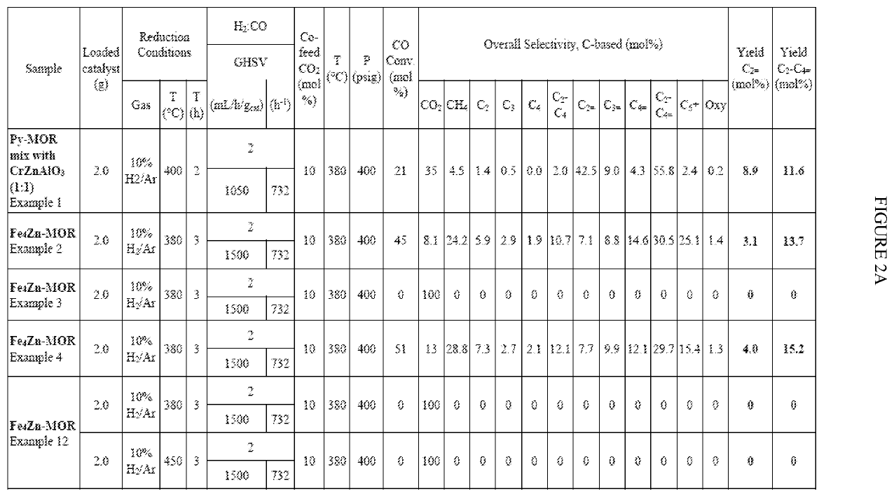

The NH₄-MOR was impregnated with iron and zinc through a solid ion exchange reaction. NH₄-MOR (8.00 g) was loaded into a large mortar with FeCl₂·4H₂O (0.85 g, 4.30 mmol) and Zn(NO₃)₂·6H₂O (0.32, 1.07 mmol). The solids were ground with a pestle until a homogeneous fine powder was obtained. Half the sample (4 g) was transferred to a quartz boat and loaded into the split tube furnace. The split tube furnace was purged with purified nitrogen for 3 hours at a flow rate of 85 sccm. The flow rate was reduced to 30 sccm and the furnace was heated to 150° C. with a 1-hour ramp. The temperature was held at 150° C. for 1 hour to allow the hydrated salts to melt and liquify. The temperature was then ramped to 400° C. at a rate of 1° C./minute and calcined at 400° C. for 4 hours. There was 3.38 g of material recovered, the off-white powder speckled with small orange dots. The FeZn₍ₛ₎—H-MOR powder was pressed with 12 metric tons using a 1-inch die set, crushed using a mortar and pestle, then sieved to 500-710 μm. X-ray Diffraction (XRD) was used to confirm that the Mordenite structure and pores were still intact. The elemental concentrations, as determined by X-ray fluorescence (XRF) on a Bruker Tracer 5G are shown in FIG. 1A.

### Example 3: Synthesis of FeZn₍ₛ₎—H-MOR—2

Na-MOR was converted to NH₄-MOR through three consecutive aqueous ions exchange reactions. Two separate batches were executed in parallel. To two 2-L RBFs was charged 30 g of Na-MOR and 1.5 L of 1 M NH₄Cl solution. The contents of the RBFs were stirred at 80° C. for 3 hours. The solutions were then filtered into their own respective Buchner funnels containing a single layer of quantitative filter paper. Each filter cake was washed with 1 L of distilled water and dried in an oven at 90° C. overnight. The yields were 26.72 g and 26.46 g respectively. To two 2-L RBFs was charged 26.72 g of Na/NH₄-MOR with 1.336 L of 1 M NH₄Cl solution and 26.46 g of Na/NH₄-MOR with 1.323 L of 1 M NH₄Cl solution respectively. The contents of the RBF were stirred at 80° C. for 3.2 hours. The solutions were then filtered into their own respective Buchner funnels containing a single layer of quantitative filter paper. Each filter cake was washed with 1 L of distilled water and dried in an oven at 90° C. overnight. The yields were 26.29 g and 26.35 g respectively. To two 2-L RBFs was charged 26.29 g of Na/NH₄-MOR with 1.315 L of 1 M NH₄Cl solution and 26.35 g of Na/NH₄-MOR with 1.318 L of 1 M NH₄Cl solution respectively. The contents of the RBF were stirred at 80° C. for 3 hours. The solutions were then filtered into their own respective Buchner funnels containing a single layer of quantitative filter paper. Each filter cake was washed with 1 L of distilled water and dried in an oven at 90° C. overnight. The yields were 26.36 g and 25.83 g respectively. Both batches were combined and thoroughly mixed.

The NH₄-MOR was impregnated with iron and zinc through a solid ion exchange reaction. NH₄-MOR (8.00 g) was loaded into a large mortar with FeCl₂·4H₂O (0.85 g, 4.30 mmol) and Zn(NO₃)₂·6H₂O (0.32, 1.07 mmol). The solids were ground with a pestle until a homogeneous fine powder was obtained. The sample was transferred to a quartz boat and loaded into the split tube furnace. The split tube furnace was purged with purified nitrogen for 1 hour at a flow rate of 85 sccm. The flow rate was reduced to 30 sccm and the furnace was heated to 150° C. with a 1-hour ramp. The temperature was held at 150° C. for 1 hour to allow the hydrated salts to melt and liquify. The temperature was then ramped to 400° C. over 2.5 hours and calcined at 400° C. for 4 hours. There was 7.97 g of material recovered, the off-white powder speckled with small orange dots. The FeZn₍ₛ₎—H-MOR powder was pressed with 12 metric tons using a 1-inch die set, crushed using a mortar and pestle, then sieved to 500-710 μm. The elemental concentrations, as determined by X-ray fluorescence (XRF) are shown in FIG. 1A.

### Example 4: Synthesis of FeZn₍ₛ₎—H-MOR—3

Na-MOR was converted to NH₄-MOR through three consecutive aqueous ions exchange reactions. Two separate batches were executed in parallel. To two 2-L RBFs was charged 30 g of Na-MOR and 1.5 L of 1 M NH₄Cl solution. The contents of the RBFs were stirred at 80° C. for 3 hours. The solutions were then filtered into their own respective Buchner funnels containing a single layer of quantitative filter paper. Each filter cake was washed with 1 L of distilled water and dried in an oven at 90° C. overnight. The yields were 26.72 g and 26.46 g respectively. To two 2-L RBFs was charged 26.72 g of Na/NH₄-MOR with 1.336 L of 1 M NH₄Cl solution and 26.46 g of Na/NH₄-MOR with 1.323 L of 1 M NH₄Cl solution respectively. The contents of the RBF were stirred at 80° C. for 3.2 hours. The solutions were then filtered into their own respective Buchner funnels containing a single layer of quantitative filter paper. Each filter cake was washed with 1 L of distilled water and dried in an oven at 90° C. overnight. The yields were 26.29 g and 26.35 g respectively. To two 2-L RBFs was charged 26.29 g of Na/NH₄-MOR with 1.315 L of 1 M NH₄Cl solution and 26.35 g of Na/NH₄-MOR with 1.318 L of 1 M NH₄Cl solution respectively. The contents of the RBF were stirred at 80° C. for 3 hours. The solutions were then filtered into their own respective Buchner funnels containing a single layer of quantitative filter paper. Each filter cake was washed with 1 L of distilled water and dried in an oven at 90° C. overnight. The yields were 26.36 g and 25.83 g respectively. Both batches were combined and thoroughly mixed.

The NH₄-MOR was impregnated with iron and zinc through a solid ion exchange reaction. NH₄-MOR (10 g) was loaded into a large mortar with FeCl₂·4H₂O (1.07 g, 5.37 mmol) and Zn(NO₃)₂·6H₂O (0.40, 1.34 mmol). The solids were ground with a pestle until a homogeneous fine powder was obtained. A subsample (4.41 g) was transferred to a quartz boat and loaded into the split tube furnace. The split tube furnace was purged with purified nitrogen for 1 hour at a flow rate of 85 sccm. The flow rate was reduced to 30 sccm and the furnace was heated to 150° C. with a 1-hour ramp. The temperature was held at 150° C. for 1 hour to allow the hydrated salts to melt and liquify. The temperature was then ramped to 400° C. over 2.5 hours and calcined at 400° C. for 4 hours. There was 3.77 g of material recovered, the off-white powder speckled with small orange dots. The FeZn₍ₛ₎—H-MOR powder was pressed with 12 metric tons using a 1-inch die set, crushed using a mortar and pestle, then sieved to 500-710 μm.

### Example 5: Synthesis of FeZn_{(aq)}—H-MOR

Na-MOR was converted to NH₄-MOR through three consecutive aqueous ions exchange reactions. To a 2-L RBF was loaded 30 g of Na-MOR and 1.5 L of 1 M NH₄Cl solution. The contents of the RBF were stirred at 80° C. for 3 hours. The solution was then filtered into a Buchner funnel containing three qualitative filter papers. The filter cake was washed with 500 mL of distilled water and dried in an oven at 90° C. overnight. There was 26.46 g of material recovered. To a 2-L RBF was loaded 26.46 g of Na/NH₄-MOR and 1.325 L of 1 M NH₄Cl solution. The contents of the RBF were stirred at 80° C. for 3 hours. The solution was then filtered into a Buchner funnel containing three qualitative filter papers. The filter cake was washed with two 500-mL portions of distilled water and dried in an oven at 90° C. overnight. There was 25.48 g of material recovered. To a 2-L RBF was loaded 25.48 g of Na/NH₄-MOR and 1.275 L of 1 M NH₄Cl solution. The contents of the RBF were stirred at 80° C. for 3 hours. The solution was then filtered into a Buchner funnel containing three qualitative filter papers. The filter cake was washed with three 500-mL portions of distilled water and dried in an oven at 90° C. overnight. The material was left uncalcined as NH₄-MOR.

The NH₄-MOR was impregnated with iron and zinc through an aqueous ion exchange reaction. FeCl₂·4H₂O (12.72 g, 63.89 mmol) and Zn(NO₃)₂·6H₂O (4.76, 16.00 mmol) were dissolved in 400 mL of distilled water in a 1-L RBF. To the solution was added 7.8 g of NH₄-MOR. The contents of the RBF were stirred at 80° C. for 3 hours. The solution was then filtered into a Buchner funnel containing three qualitative filter papers. The filter cake was washed with 500 mL of distilled water and dried in an oven at 90° C. overnight. There was 7.68 g of material recovered and it was a light peach color. Half the sample (3.81 g) was loaded into a quartz boat. The quartz boat was loaded into the split tube furnace and purged with purified nitrogen for 1.5 hours at a flow rate of 85 sccm. The flow rate was reduced to 30 sccm and the furnace was heated to 400° C. at a ramp rate of 1° C./minute. The sample was calcined 400° C. for 4 hours and 3.48 g of material was recovered. The FeZn_{(aq)}—H-MOR powder was pressed with 12 metric tons using a 1-inch die set, crushed using a mortar and pestle, then sieved to 500-710 μm. The elemental concentrations, as determined by X-ray fluorescence (XRF) are shown in FIG. 1A.

### Example 6: Synthesis of 2×FeZn₍ₛ₎—H-MOR

Na-MOR was converted to NH₄-MOR through three consecutive aqueous ions exchange reactions. Two separate batches were executed in parallel. To two 2-L RBFs was charged 30 g of Na-MOR and 1.5 L of 1 M NH₄Cl solution. The contents of the RBFs were stirred at 80° C. for 3 hours. The solutions were then filtered into their own respective Buchner funnels containing a single layer of quantitative filter paper. Each filter cake was washed with 1 L of distilled water and dried in an oven at 90° C. overnight. The yields were 26.72 g and 26.46 g respectively. To two 2-L RBFs was charged 26.72 g of Na/NH₄-MOR with 1.336 L of 1 M NH₄Cl solution and 26.46 g of Na/NH₄-MOR with 1.323 L of 1 M NH₄Cl solution respectively. The contents of the RBF were stirred at 80° C. for 3.2 hours. The solutions were then filtered into their own respective Buchner funnels containing a single layer of quantitative filter paper. Each filter cake was washed with 1 L of distilled water and dried in an oven at 90° C. overnight. The yields were 26.29 g and 26.35 g respectively. To two 2-L RBFs was charged 26.29 g of Na/NH₄-MOR with 1.315 L of 1 M NH₄Cl solution and 26.35 g of Na/NH₄-MOR with 1.318 L of 1 M NH₄Cl solution respectively. The contents of the RBF were stirred at 80° C. for 3 hours. The solutions were then filtered into their own respective Buchner funnels containing a single layer of quantitative filter paper. Each filter cake was washed with 1 L of distilled water and dried in an oven at 90° C. overnight. The yields were 26.36 g and 25.83 g respectively. Both batches were combined and thoroughly mixed.

The NH₄-MOR was impregnated with iron and zinc through a solid ion exchange reaction. NH₄-MOR (8 g) was loaded into a large mortar with FeCl₂·4H₂O (1.71 g, 8.59 mmol) and Zn(NO₃)₂·6H₂O (0.64, 1.24 mmol). The solids were ground with a pestle until a homogeneous fine powder was obtained. The sample was transferred to a quartz boat and loaded into the split tube furnace. The split tube furnace was purged with purified nitrogen for 1 hour at a flow rate of 85 sccm. The flow rate was reduced to 30 sccm and the furnace was heated to 150° C. with a 1-hour ramp. The temperature was held at 150° C. for 1 hour to allow the hydrated salts to melt and liquify. The temperature was then ramped to 400° C. over 2.5 hours and calcined at 400° C. for 4 hours. There was 8.30 g of material recovered, the off-white powder speckled with small orange dots. A subsample, roughly half, of the FeZn₍ₛ₎—H-MOR powder was pressed with 12 metric tons using a 1-inch die set, crushed using a mortar and pestle, then sieved to 500-710 μm. The elemental concentrations, as determined by X-ray fluorescence (XRF) are shown in FIG. 1B.

### Example 7: Synthesis of FeZn₍ₛ₎—H-MOR—Alternative Starting Material

Na-MOR was converted to NH₄-MOR through three consecutive aqueous ions exchange reactions. To two 2-L RBFs was charged 30 g of Na-MOR and 1.5 L of 1 M NH₄Cl solution. The contents of the RBFs were stirred at 80° C. for 4 hours. The solutions were then filtered into a single Buchner funnel containing three layers of qualitative filter papers. The filter cake was washed with 1 L of distilled water and dried in an oven at 90° C. overnight. The yield was 54.05 g. The Na/NH₄-MOR was separated into two 27 g batches and these were loading into two separate 2-L RBFs with 1.35 L of 1 M NH₄Cl solution. The contents of the RBF were stirred at 80° C. for 4 hours. The solutions were then filtered into their own respective Buchner funnels containing a single layer of quantitative filter paper. Each filter cake was washed with 1 L of distilled water and dried in an oven at 90° C. overnight. The yields were 25.9 g and 25.8 g respectively. To two 2-L RBFs was charged 25.9 g of Na/NH₄-MOR with 1.3 L of 1 M NH₄Cl solution and 25.8 g of Na/NH₄-MOR with 1.29 L of 1 M NH₄Cl solution respectively. The contents of the RBF were stirred at 80° C. for 4 hours. The solutions were then filtered into their own respective Buchner funnels containing a single layer of quantitative filter paper. Each filter cake was washed with 1 L of distilled water and dried in an oven at 90° C. overnight. Both batches were combined and thoroughly mixed.

The NH₄-MOR was impregnated with iron and zinc through a solid ion exchange reaction. NH₄-MOR (4.00 g) was loaded into a large mortar with FeCl₂·4H₂O (0.44 g, 2.20 mmol) and Zn(OAc)₂·6H₂O (0.12, 0.54 mmol). The solids were ground with a pestle until a homogeneous fine powder was obtained. The sample was transferred to a quartz boat and loaded into the split tube furnace. The split tube furnace was purged with purified nitrogen for 1 hour at a flow rate of 85 sccm. The flow rate was reduced to 30 sccm and the furnace was heated to 260° C. with a 1-hour ramp. The temperature was held at 260° C. for 1 hour to allow the hydrated salts to melt and liquify. The temperature was then ramped to 400° C. over 2.5 hours and calcined at 400° C. for 4 hours. There was 4.00 g of material recovered. The FeZn₍ₛ₎—H-MOR powder was pressed with 12 metric tons using a 1-inch die set, crushed using a mortar and pestle, then sieved to 500-710 μm. The elemental concentrations, as determined by X-ray fluorescence (XRF) are shown in FIG. 1B.

### Example 8: Synthesis of FeZn₍ₛ₎—H-MOR—High Temp Calcination

Na-MOR was converted to NH₄-MOR through three consecutive aqueous ions exchange reactions. To two 2-L RBFs was charged 30 g of Na-MOR and 1.5 L of 1 M NH₄Cl solution. The contents of the RBFs were stirred at 80° C. for 4 hours. The solutions were then filtered into a single Buchner funnel containing three layers of qualitative filter papers. The filter cake was washed with 1 L of distilled water and dried in an oven at 90° C. overnight. The yield was 54.05 g. The Na/NH₄-MOR was separated into two 27 g batches and these were loading into two separate 2-L RBFs with 1.35 L of 1 M NH₄Cl solution. The contents of the RBF were stirred at 80° C. for 4 hours. The solutions were then filtered into their own respective Buchner funnels containing a single layer of quantitative filter paper. Each filter cake was washed with 1 L of distilled water and dried in an oven at 90° C. overnight. The yields were 25.9 g and 25.8 g respectively. To two 2-L RBFs was charged 25.9 g of Na/NH₄-MOR with 1.3 L of 1 M NH₄Cl solution and 25.8 g of Na/NH₄-MOR with 1.29 L of 1 M NH₄Cl solution respectively. The contents of the RBF were stirred at 80° C. for 4 hours. The solutions were then filtered into their own respective Buchner funnels containing a single layer of quantitative filter paper. Each filter cake was washed with 1 L of distilled water and dried in an oven at 90° C. overnight. Both batches were combined and thoroughly mixed.

The NH₄-MOR was impregnated with iron and zinc through a solid ion exchange reaction. NH₄-MOR (4.00 g) was loaded into a large mortar with FeCl₂·4H₂O (0.85 g, 4.30 mmol) and Zn(NO₃)₂·6H₂O (0.32, 1.07 mmol). The solids were ground with a pestle until a homogeneous fine powder was obtained. The sample was transferred to a quartz boat and loaded into the split tube furnace. The split tube furnace was purged with purified nitrogen for 3 hours at a flow rate of 85 sccm. The flow rate was reduced to 30 sccm and the furnace was heated to 150° C. with a 1-hour ramp. The temperature was held at 150° C. for 1 hour to allow the hydrated salts to melt and liquify. The temperature was then ramped to 600° C. at a rate of 1° C./minute and calcined at 600° C. for 4 hours. There was 4.07 g of material recovered, the off-white powder speckled with small dark/black specs. The FeZn₍ₛ₎—H-MOR powder was pressed with 12 metric tons using a 1-inch die set, crushed using a mortar and pestle, then sieved to 500-710 μm. The elemental concentrations, as determined by X-ray fluorescence (XRF) are shown in FIG. 1B.

### Example 9: Synthesis of FeZn₍ₛ₎—H-MOR

Na-MOR was converted to NH4-MOR through three consecutive aqueous ions exchange reactions. To two 2-L RBFs was charged 30 g of Na-MOR and 1.5 L of 1 M NH₄Cl solution. The contents of the RBFs were stirred at 80° C. for 4 hours. The solutions were then filtered into a single Buchner funnel containing three layers of qualitative filter papers. The filter cake was washed with 1 L of distilled water and dried in an oven at 90° C. overnight. The yield was 54.05 g. The Na/NH₄-MOR was separated into two 27 g batches and these were loading into two separate 2-L RBFs with 1.35 L of 1 M NH₄Cl solution. The contents of the RBF were stirred at 80° C. for 4 hours. The solutions were then filtered into their own respective Buchner funnels containing a single layer of quantitative filter paper. Each filter cake was washed with 1 L of distilled water and dried in an oven at 90° C. overnight. The yields were 25.9 g and 25.8 g respectively. To two 2-L RBFs was charged 25.9 g of Na/NH₄-MOR with 1.3 L of 1 M NH₄Cl solution and 25.8 g of Na/NH₄-MOR with 1.29 L of 1 M NH₄Cl solution respectively. The contents of the RBF were stirred at 80° C. for 4 hours. The solutions were then filtered into their own respective Buchner funnels containing a single layer of quantitative filter paper. Each filter cake was washed with 1 L of distilled water and dried in an oven at 90° C. overnight. Both batches were combined and thoroughly mixed.

The NH₄-MOR was impregnated with iron and zinc through a solid ion exchange reaction. NH₄-MOR (4.00 g) was loaded into a large mortar with FeCl₂·4H₂O (0.48 g, 2.40 mmol) and Zn(NO₃)₂·6H₂O (0.16, 0.53 mmol). The solids were ground with a pestle until a homogeneous fine powder was obtained. The sample was transferred to a quartz boat and loaded into the split tube furnace. The split tube furnace was purged with purified nitrogen for 3 hours at a flow rate of 85 sccm. The flow rate was reduced to 30 sccm and the furnace was heated to 150° C. with a 1-hour ramp. The temperature was held at 150° C. for 1 hour to allow the hydrated salts to melt and liquify. The temperature was then ramped to 400° C. at a rate of 1° C./minute and calcined at 400° C. for 4 hours. There was 3.96 g of material recovered. The FeZn₍ₛ₎—H-MOR powder was pressed with 12 metric tons using a 1-inch die set, crushed using a mortar and pestle, then sieved to 500-710 μm. The elemental concentrations, as determined by X-ray fluorescence (XRF) are shown in FIG. 1B.

### Example 10: Synthesis of FeZn₍ₛ₎—H-MOR—Alternative Starting Material

Na-MOR was converted to NH₄-MOR through three consecutive aqueous ions exchange reactions. To two 2-L RBFs was charged 30 g of Na-MOR and 1.5 L of 1 M NH₄Cl solution. The contents of the RBFs were stirred at 80° C. for 4 hours. The solutions were then filtered into a single Buchner funnel containing three layers of qualitative filter papers. The filter cake was washed with 1 L of distilled water and dried in an oven at 90° C. overnight. The yield was 54.05 g. The Na/NH₄-MOR was separated into two 27 g batches and these were loading into two separate 2-L RBFs with 1.35 L of 1 M NH₄Cl solution. The contents of the RBF were stirred at 80° C. for 4 hours. The solutions were then filtered into their own respective Buchner funnels containing a single layer of quantitative filter paper. Each filter cake was washed with 1 L of distilled water and dried in an oven at 90° C. overnight. The yields were 25.9 g and 25.8 g respectively. To two 2-L RBFs was charged 25.9 g of Na/NH₄-MOR with 1.3 L of 1 M NH₄Cl solution and 25.8 g of Na/NH₄-MOR with 1.29 L of 1 M NH₄Cl solution respectively. The contents of the RBF were stirred at 80° C. for 4 hours. The solutions were then filtered into their own respective Buchner funnels containing a single layer of quantitative filter paper. Each filter cake was washed with 1 L of distilled water and dried in an oven at 90° C. overnight. Both batches were combined and thoroughly mixed.

The NH₄-MOR was impregnated with iron and zinc through a solid ion exchange reaction. NH₄-MOR (4.00 g) was loaded into a large mortar with FeSO₄·7H₂O (1.19 g, 4.30 mmol) and Zn(NO₃)₂·6H₂O (0.32, 1.07 mmol). The solids were ground with a pestle until a homogeneous fine powder was obtained. The sample was transferred to a quartz boat and loaded into the split tube furnace. The split tube furnace was purged with purified nitrogen for 3 hours at a flow rate of 85 sccm. The flow rate was reduced to 30 sccm and the furnace was heated to 150° C. with a 1-hour ramp. The temperature was held at 150° C. for 1 hour to allow the hydrated salts to melt and liquify. The temperature was then ramped to 400° C. at a rate of 1° C./minute and calcined at 400° C. for 4 hours. The FeZn₍ₛ₎—H-MOR powder was pressed with 12 metric tons using a 1-inch die set, crushed using a mortar and pestle, then sieved to 500-710 μm. The elemental concentrations, as determined by X-ray fluorescence (XRF) are shown in FIG. 1B.

### Example 11: Synthesis of FeZn₍ₛ₎—H-MOR—Alternative Starting Material

Na-MOR was converted to NH₄-MOR through three consecutive aqueous ions exchange reactions. To two 2-L RBFs was charged 30 g of Na-MOR and 1.5 L of 1 M NH₄Cl solution. The contents of the RBFs were stirred at 80° C. for 4 hours. The solutions were then filtered into a single Buchner funnel containing three layers of qualitative filter papers. The filter cake was washed with 1 L of distilled water and dried in an oven at 90° C. overnight. The yield was 54.05 g. The Na/NH₄-MOR was separated into two 27 g batches and these were loading into two separate 2-L RBFs with 1.35 L of 1 M NH₄Cl solution. The contents of the RBF were stirred at 80° C. for 4 hours. The solutions were then filtered into their own respective Buchner funnels containing a single layer of quantitative filter paper. Each filter cake was washed with 1 L of distilled water and dried in an oven at 90° C. overnight. The yields were 25.9 g and 25.8 g respectively. To two 2-L RBFs was charged 25.9 g of Na/NH₄-MOR with 1.3 L of 1 M NH₄Cl solution and 25.8 g of Na/NH₄-MOR with 1.29 L of 1 M NH₄Cl solution respectively. The contents of the RBF were stirred at 80° C. for 4 hours. The solutions were then filtered into their own respective Buchner funnels containing a single layer of quantitative filter paper. Each filter cake was washed with 1 L of distilled water and dried in an oven at 90° C. overnight. Both batches were combined and thoroughly mixed.

The NH₄-MOR was impregnated with iron and zinc through a solid ion exchange reaction. NH₄-MOR (4.00 g) was loaded into a large mortar with Fe(NO₃)₂·9H₂O (1.74 g, 4.28 mmol) and Zn(NO₃)₂·6H₂O (0.32, 1.07 mmol). The solids were ground with a pestle until a homogeneous fine powder was obtained. The sample was transferred to a quartz boat and loaded into the split tube furnace. The split tube furnace was purged with purified nitrogen for 1 hour at a flow rate of 85 sccm. The flow rate was reduced to 30 sccm and the furnace was heated to 150° C. with a 1-hour ramp. The temperature was held at 150° C. for 1 hour to allow the hydrated salts to melt and liquify. The temperature was then ramped to 400° C. over 2.5 hours and calcined at 400° C. for 4 hours. There was 7.97 g of material recovered, the off-white powder speckled with small orange dots. The elemental concentrations, as determined by X-ray fluorescence (XRF) are shown in FIG. 1B.

### Example 12: Synthesis of Synthesis of FeZn₍ₛ₎—H-MOR—4

Na-MOR was converted to NH₄-MOR through three consecutive aqueous ions exchange reactions. Two separate batches were executed in parallel. To two 2-L RBFs was charged 30 g of Na-MOR and 1.5 L of 1 M NH₄Cl solution. The contents of the RBFs were stirred at 80° C. for 3 hours. The solutions were then filtered into their own respective Buchner funnels containing a single layer of quantitative filter paper. Each filter cake was washed with 1 L of distilled water and dried in an oven at 90° C. overnight. The yields were 26.72 g and 26.46 g respectively. To two 2-L RBFs was charged 26.72 g of Na/NH₄-MOR with 1.336 L of 1 M NH₄Cl solution and 26.46 g of Na/NH₄-MOR with 1.323 L of 1 M NH₄Cl solution respectively. The contents of the RBF were stirred at 80° C. for 3.2 hours. The solutions were then filtered into their own respective Buchner funnels containing a single layer of quantitative filter paper. Each filter cake was washed with 1 L of distilled water and dried in an oven at 90° C. overnight. The yields were 26.29 g and 26.35 g respectively. To two 2-L RBFs was charged 26.29 g of Na/NH₄-MOR with 1.315 L of 1 M NH₄Cl solution and 26.35 g of Na/NH₄-MOR with 1.318 L of 1 M NH₄Cl solution respectively. The contents of the RBF were stirred at 80° C. for 3 hours. The solutions were then filtered into their own respective Buchner funnels containing a single layer of quantitative filter paper. Each filter cake was washed with 1 L of distilled water and dried in an oven at 90° C. overnight. The yields were 26.36 g and 25.83 g respectively. Both batches were combined and thoroughly mixed. The elemental concentrations, as determined by X-ray fluorescence (XRF) are shown in FIG. 1B.

The NH₄-MOR was impregnated with iron and zinc through a solid ion exchange reaction. NH₄-MOR (8.00 g) was loaded into a large mortar with FeCl₂·4H₂O (0.85 g, 4.30 mmol) and Zn(NO₃)₂·6H₂O (0.32, 1.07 mmol). The solids were ground with a pestle until a homogeneous fine powder was obtained. A subsample (4.13 g) was transferred to a quartz boat and loaded into the split tube furnace. The split tube furnace was purged with purified nitrogen for 1 hour at a flow rate of 85 sccm. The flow rate was reduced to 30 sccm and the furnace was heated to 150° C. with a 1-hour ramp. The temperature was held at 150° C. for 1 hour to allow the hydrated salts to melt and liquify. The temperature was then ramped to 400° C. over 2.5 hours and calcined at 400° C. for 4 hours. There was 3.56 g of material recovered, the off-white powder speckled with small orange dots. The Fe₄Zn-MOR powder was pressed with 12 metric tons using a 1-inch die set, crushed using a mortar and pestle, then sieved to 500-710 μm. The elemental concentrations, as determined by X-ray fluorescence (XRF) are shown in FIG. 1A.

### Example 13: Catalyst Testing

### Equipment

The CO and CO₂ hydrogenation in presence of a gas with 56% by volume H₂, 28% by volume CO, 10% by volume CO₂ and 6% by volume Ar with a GHSV of 1500 mL/h/g_{cat} for the overall gas feed was accomplished in a fixed-bed syngas converter unit (SCU) built in-house. The SCU included a packed bed tubular reactor housed in a furnace with a single heating zone. The reactor tube was made from SS316 stainless-steel (Swagelok) which had an outer diameter of 0.5 inches, an internal diameter of about 0.4 inches, and a length of about 22 inches. The reactor was heated using a WATLOW heater equipped with a temperature limit controller. The thermocouple (K-type) having an outer diameter of 1/16 (0.0625) inches was inserted axially through the center of the reactor, which was used to measure and control the temperature within the catalyst bed of approximately 50 mm height.

### Catalyst Loading

The particle size of the catalyst used were in the range of 0.71 mm to 0.5 mm. No diluents of any kind were used to prepare the catalysts prior to catalytic testing. The catalyst was housed on top of glass beads (Fischer Scientific, 5 mm size, 30 g) spaced by glass wool. Either pure α-Al₂O₃ (Sasol, 10 gram) beads (0.5-1.0 mm diameter) calcined at 1100° C. were used on either end of the reactor tube before and after the catalyst bed and spaced by the glass wool or glass beads were used below the catalyst bed at the bottom of the reactor tube. In total, the whole length of the reactor tube was filled up (approximately 20 inches) with inert materials to minimize the temperature gradient.

### Testing Procedure

In order to approach plug flow conditions and minimize back mixing and channeling, certain operating criteria such as the ratio of catalyst bed length to catalyst particle size (L/Dₚ) was maintained at more than 50 and the ratio of the inside diameter of the reactor to catalyst particle size (D/Dₚ) was maintained at more than 10. Prior to each experimental run for catalyst evaluation, the catalyst was activated by in situ reduction at 380-450° C. for 2-15 hours by flowing 10% H₂in Ar (Linde) using a mass flow controller (Bronkhorst) at atmospheric pressure. The catalyst test was accomplished at temperature ranging from 350° C. to 380° C. Pressure was also varied from 300 to 400 psig. A premixed gas mixture (H₂/CO volume ratio 2 with 10% CO₂ by volume (Linde) was used as a feed. The gas hourly space velocity (GHSV) dictated the volume of gas flow rate depending on the volume of catalyst used in the experiment. Typically, the catalyst amount used was 2.0 grams at a given flow rate. GHSV was defined as volumetric flow of the reactor feed gas divided by the volume of the catalyst bed. The GHSV in mL/h/g_{cat} was calculated as

$$GHSV = \frac{Volumetric flow rate ( or feed flow rate )}{mass of the catalyst}$$

When using units of h⁻¹, the GHSV is calculated as

$$GHSV = \frac{Volumetric flow rate ( or feed flow rate )}{volume of the catalyst}$$

The feed and product gases were analyzed with an on-line gas chromatograph (7890B, Agilent Technologies). The GC was equipped with 3 detectors. The front flame ionization detector (FID) detected hydrocarbons from C1 to C9 and also separated ethane, ethylene, propane, propylene, butane, and butylene using an Alumina Plot column. The heavier hydrocarbons like aromatics (benzene, toluene, ethylbenzene, p-xylene, o-xylene, m-xylene), oxygenates (methanol, ethanol, and acetones etc.) were detected on another FID which used a CP Wax57 column. The permanent gases (H₂, O₂/Ar, N₂, CH₄, CO, CO₂) were detected on a TCD (thermal conductivity detector) and separated on a Haysep and molecular sieve column.

A chilled water condenser (Lauda chiller, operating at 5° C.) was located after the reactor to collect heavier hydrocarbon and water condensates. The total gas volumetric flow rate after the reaction was calculated based on Ar that was used as an internal standard in the feed mixture. The conversion of CO and selectivities of CO₂ and C₂-C₄ olefins were calculated as described above.

The selectivities of ethylene only (S_{C}_{₂}_{H}_{₄}(%)), paraffins (S_{C}_{₂}_{-C}_{₄}₋( %)), C₅₊(S_{C}_{₅}₊ (%)) and methane (S_{CH}_{₄} (%)) were calculated as follows:

$$\begin{matrix}{S}_{{C}_{2} {H}_{4}} ( \% ) = \frac{{n}_{{C}_{2} {H}_{4}}}{{n}_{CO} , in - {n}_{CO} , out} \times 100 \\ {S}_{{C}_{2} - {C}_{4} =} ( \% ) = \frac{{n}_{{C}_{2} {H}_{6}} + {n}_{{C}_{3} {H}_{8}} + {n}_{{C}_{4} {H}_{10}}}{{n}_{CO} , in - {n}_{CO} , out} \times 100 \\ {S}_{{C}_{5} +} ( \% ) = \frac{{n}_{{C}_{5} +}}{{n}_{CO} , in - {n}_{CO} , out} \times 100 \\ {S}_{{CH}_{4}} ( \% ) = \frac{{n}_{{CH}_{4}}}{{n}_{CO} , in - {n}_{CO} , out} \times 100 \\ {Y}_{{C}_{2} =} ( mol \% ) = {S}_{{C}_{2} =} \times {X}_{CO} \div 100 \\ {Y}_{{C}_{2} - {C}_{4} =} ( mol \% ) = {S}_{{C}_{2} - {C}_{4}} \times {X}_{CO} \div 100\end{matrix}$$

n_{CO}, in is the moles of CO input. n_{CO}, out is the moles of CO output. n_{C}_{₂}_{H}_{₄} is the moles of C₂H₄ output. n_{C}_{₂}_{H}_{₆} is the moles of C₂H₆ output. n_{C}_{₃}_{H}_{₈} is the moles of C₃H₈ output. n_{C}_{₄}_{H}_{₁₀} is the moles of C₄H₁₀ output. n_{CH}_{₄} is the moles of CH₄ output. n_{C}_{₅₊} is the moles of C₅₊ output.

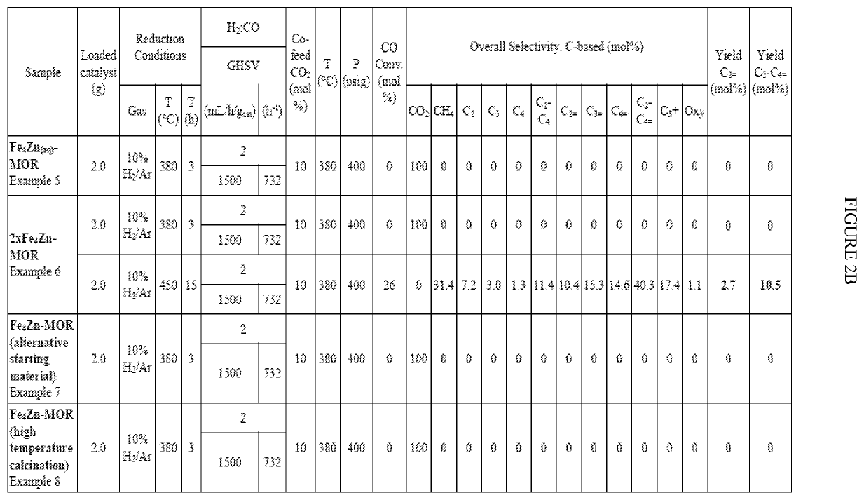

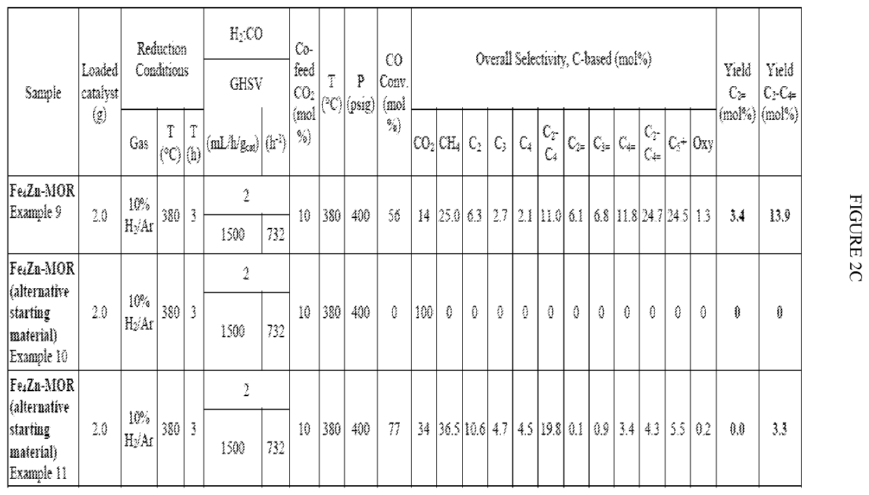

For all reporting data, the carbon balances were higher than 95%. And the selectivities were normalized to 100. The experimental conditions, CO conversion and product selectivities are reported in FIGS. 2A-2C.

### Other Embodiments

While certain embodiments have been described, the disclosure is not limited to such embodiments.

As an example, although making the catalyst via a solid-state ion exchange process has been disclosed, the disclosure is not limited to such processes. For example, the catalyst according to the disclosure can be made using incipient wetness impregnation or atomic layer deposition.

As another example, while use of a catalyst in a syngas reaction has been described, the disclosure is not limited in this sense. For example, the catalyst according to the disclosure can be used in the conversion of methanol to olefins or dimethyl ether carbonylation to methyl acetate.

1. A catalyst, comprising:
H-MOR;
from 2.0 weight percent (wt %) to 6.5 wt % Fe; and
from 0.1 wt % to 2.0 wt % Zn;
wherein the catalyst comprises a Fe/Zn molar ratio of from 3.25 to 5.0.

2.-77. (canceled)

78. The catalyst of claim 1, wherein the catalyst has a Fe/Zn molar ratio of from 3.75 to 5.0.

79. The catalyst of claim 1, wherein the catalyst has a Fe/Zn molar ratio of from 3.8 to 4.5.

80. The catalyst of claim 1, wherein the catalyst has a Fe/Zn molar ratio of from 3.9 to 4.5.

81. The catalyst of claim 1, wherein the catalyst comprises from 0.2 wt % to 1.8 wt % Zn.

82. The catalyst of claim 1, wherein the catalyst comprises from 0.3 wt % to 1.75 wt % Zn.

83. The catalyst of claim 1, wherein the catalyst comprises from 0.4 wt % to 1.7 wt % Zn.

84. The catalyst of claim 1, wherein the catalyst comprises from 1.0 wt % to 6.0 wt % Fe.

85. The catalyst of claim 1, wherein the catalyst comprises from 2.3 wt % to 5.8 wt % Fe.

86. The catalyst of claim 1, wherein the catalyst comprises from 2.8 wt % to 5.6 wt % Fe.

87. The catalyst of claim 1, wherein at least one of the following holds:
the catalyst has a CO₂ selectivity of at most 30%;
the catalyst has a CO conversion of at least 40%; or
the catalyst has a selectivity for C₂-C₄ olefins of at least 20%.

88. A method, comprising:
contacting a gas mixture comprising H₂, CO and CO₂ and a catalyst to form C₂-C₄ olefins,
wherein the catalyst is a catalyst according to claim 1.

89. A catalyst, comprising:
H-MOR;
Fe; and
Zn;
wherein the catalyst has a Fe/Zn molar ratio of from 3.5 to 5.0.

90. The catalyst of claim 89, wherein the catalyst has a Fe/Zn molar ratio of from 3.75 to 5.0.

91. The catalyst of claim 89, wherein the catalyst has a Fe/Zn molar ratio of from 3.8 to 4.5.

92. The catalyst of claim 89, wherein the catalyst has a Fe/Zn molar ratio of from 3.9 to 4.5.

93. The catalyst of claim 89, wherein the catalyst comprises from 2.0 weight percent (wt %) to 6.5 wt % Fe.

94. The catalyst of claim 89, wherein the catalyst comprises from 0.1 wt % to 2.0 wt % Zn.

95. The catalyst of claim 89, wherein at least one of the following holds:
the catalyst has a CO₂ selectivity of at most 30%;
the catalyst has a CO conversion of at least 40%; or
the catalyst has a selectivity for C₂-C₄ olefins of at least 20%.

96. A method, comprising:
contacting a gas mixture comprising H₂, CO and CO₂ and a catalyst to form C₂-C₄ olefins,
wherein the catalyst is a catalyst according to claim 89.
